(19) United States
(12) Patent Application Publication
(10) Pub. No.: US 2025/0140420 A1
(43) Pub. Date: May 1, 2025

(54) SYSTEMS AND METHODS FOR PROVIDING PERSONALIZED HEALTH RISK ASSESSMENTS AND RECOMMENDATIONS

(71) Applicant: Aktivolabs Pte. Ltd., Singapore (SG)

(72) Inventors: Gourab Mukherjee, Singapore (SG); David Tien Min Lai, Kingscliff (AU)

(21) Appl. No.: 18/931,823

(22) Filed: Oct. 30, 2024

Related U.S. Application Data
(60) Provisional application No. 63/546,353, filed on Oct. 30, 2023.

(51) Int. Cl. G16H 50/30; G06N 3/044; G06N 3/08; G16H 10/60; G16H 20/30; G16H 20/60; G16H 20/70

(52) U.S. Cl. CPC G16H 50/30; G06N 3/044; G06N 3/08; G16H 10/60; G16H 20/30; G16H 20/60; G16H 20/70

(57) ABSTRACT

The invention provides systems and methods for providing real-time personalized health risk assessments and recommended interventions for a user based on a unique analysis of data collected from a user's mobile and/or wearable devices.

### CROSS-REFERENCE TO RELATED APPLICATIONS

This application claims priority to, and the benefit of, U.S. Provisional Application No. 63/546,353, filed on Oct. 30, 2023, the content of which is incorporated by reference herein in its entirety.

### TECHNICAL FIELD

The disclosure relates generally to personalized health management services, and, more particularly, to a cloud-based digital health platform for providing real-time personalized health risk assessments and recommended interventions for a user based on a unique analysis of data collected from a user's mobile and/or wearable devices.

### BACKGROUND

As global life expectancy rises, many developed nations face an aging population coupled with a significant workforce shortage in the healthcare sector. In addition, the aging population has resulted in a rise in multiple chronic conditions (i.e., non-communicable diseases), such as diabetes, hypertension, and obesity, as well as various mental health issues, such as depression, which pose serious public health problems.

The demographic shift increases the demand for healthcare services that support individuals in maintaining independent lives and managing their complex and multiple chronic conditions, particularly for those who are frail or experiencing neurodivergent and cognitive impairments.

The COVID-19 (coronavirus disease 2019) pandemic, which resulted in isolation and social distancing, highlighted the vulnerability of certain populations and underscored the urgent need for innovative healthcare solutions. Social isolation worsens with age and with chronic diseases, and societies unprepared to provide aging populations with adequate assistive technologies face heightened risks.

A key component in preventing and managing chronic diseases is the modification of risk factors. Risk factor modification refers to interventions that address lifestyle factors, such as, smoking, hypertension, high cholesterol, and physical inactivity. Patients are expected to take responsibility for managing their health by modifying risk factors and adopting lifestyle changes.

Mobile health (mHealth) technologies can support these modifications by providing users with self-management tools to monitor and adjust behaviors that impact their health. For example, tracking physical activity or blood pressure can motivate users to adopt healthier habits. mHealth utilizes smartphones, wireless devices, and applications to improve healthcare services and delivery. mHealth applications (m Health “apps”) provide a non-pharmacological approach to track a wide range of biometric, behavioral, emotional, cognitive, and psychosocial data, including physical steps, mood, diet, stress, sleep, and heart rate, to name a few. These mHealth apps promise to empower individuals by giving them control over their health and encouraging proactive wellness practices.

While this responsibility of the patients is clear, it becomes especially challenging for certain patients and despite their potential, mHealth technologies present challenges. These challenges not only involve digital disparities and inequalities in internet access, insurance coverage, and educational awareness, but also extend to the users' difficulties in interpreting the biometrics data, implementing and complying with nuanced strategies to manage and monitor their health.

The fragmented nature of the data mHealth apps provide is a key challenge. For example, step counts or heart rate data alone offer only a partial view of an individual's overall health. Many users struggle to interpret and integrate these data points holistically. As such, currently available mHealth apps are limited in use and have significant drawbacks, as users may focus on isolated data points, missing the broader picture necessary for making informed health decisions, managing chronic illness, and promoting meaningful behavior changes.

### SUMMARY

The invention of the present disclosure addresses the limitations of current digital health applications by providing systems and methods to provide real-time personalized health risk assessments and recommendations for a user based on a unique analysis of data collected from a user's mobile and/or wearable devices.

More specifically, the present invention provides a cloud-based digital health platform configured to collect, harmonize, and analyze lifestyle data (gathered in real-time from a user's mobile and/or wearable devices) to generate a digital biomarker that is indicative of the user's risk in developing a health condition and/or predictive of the user's morbidity and mortality risk of a health condition. The digital biomarker is generally a single metric, based on the combined impact of daily physical activities and sleep, that indicates a given user's health risk for developing chronic diseases. The platform essentially analyzes an individual's exercise, light physical activities, sedentary behavior, and sleep, with respect to at least the individual's age and gender, into the single metric that correlates with their health status and longevity, further predicting morbidity and mortality risks based on lifestyle factors and utilizing the metric to forecast personalized health risks and outcomes.

The digital health platform utilizes data harmonization, which is an essential step in the data collection process, as it ensures concordance across different types of devices, enabling the generation of a truly representative digital biomarker for the user. The digital health platform gathers real-time data from smartphones and wearables, connecting seamlessly with popular brands like Apple Watch, Fitbit, Garmin, Oura Ring, and Whoop for automatic data transfer. The digital biomarker generated from this data can then be combined with sociodemographic population information, electronic health records, and other relevant data sources. This combined data is fed into a risk analytics engine, which uses advanced algorithms to predict personalized cross-sectional and longitudinal health risks and outcomes for non-communicable diseases with enhanced accuracy. More specifically, in an effort to continuously improve the accuracy of health risk predictions, machine learning (ML) tools are utilized to train the risk analytics engine, a model built on datasets from multiple data banks.

The digital health platform provides an interface with which a user can interact via their smartphone or personal computing device. The interface provides, among other things, personalized health assessments, daily lifestyle tracking, and engaging content to promote healthier habits, enabling individuals, corporations, and insurers to monitor health metrics effectively and make informed choices for long term well-being. For example, the interface may generally provide the digital biomarker in the form of a score, which simplifies a user's ability to evaluate their risk factors and overall health. The interface further supports self-reporting and empowers users to take preventive actions to manage their chronic disease risk. The platform interface may generally provide tools for self-monitoring and management, incorporating behavior change interventions, goal-setting, and gamification to enhance user engagement, which can further help motivate users and support better self-management of their chronic conditions. For example, the user interface may provide health scores, recommendations, and comparative analytics in a gamified format that incentivizes use by rewarding users for achieving personalized health milestones. The use of gamified features, such as scores and badges, may help users better interpret health data and manage their conditions, thereby improving their understanding of health signals and enhances adherence to healthy lifestyle and treatment plans.

Accordingly, the digital health platform is able to effectively connect various metrics, such as physical activity with sleep quality and/or emotional well-being, which is essential for a comprehensive understanding of health and well-being. As a result, the digital health platform allows for more effective patient monitoring and may reduce the need in-person visits on an already overburdened healthcare system. Through data harmonization and the use of ML models, the personalized health risk and outcome predictions provided by the digital health platform are representative of every user, regardless of the data sources, making the model highly valuable for applications such as underwriting and population health studies. In turn, digital health platform is able to outperform other currently existing mHealth apps in predicting morbidities, co-morbidities, and mortality risk. It allows timely interventions for at-risk groups, improving individual overall health outcomes, and reducing long-term healthcare costs.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a block diagram illustrating one embodiment of an exemplary system for providing real-time personalized health risk assessments and recommendations consistent with the present disclosure.

FIG. 2 is a block diagram illustrating the digital health platform of FIG. 1 in greater detail.

FIG. 3 is a block diagram illustrating inputting of reference data (i.e., training data sets) into one or more machine learning models consistent with the present disclosure.

FIG. 4 is a block diagram illustrating an exemplary computing system for implementing one or more servers for running the digital health platform consistent with the present disclosure.

FIG. 5 is a flow diagram illustrating the overall process by which data is collected and analyzed to provide personalized health risks for a given user.

FIG. 6 is a flow diagram illustrating an exemplary process by which data is collected from mobile devices (i.e., smartphones) and wearable devices (i.e., watches, wristbands, rings, etc.) and subsequently processed for data harmonization purposes.

FIG. 7 shows an exemplary embodiment of a machine learning model used for predicting various physical activities that users perform (based on data harmonization techniques so as to standardize data across smartphones and wearable devices).

FIGS. 8A-8B, 9A-9B, and 10A-10B are graphs showing Root Mean Squared Error (RMSE) calculated for use of machine learning models of the present invention versus current methods.

FIG. 11 is a flow diagram illustrating an exemplary data cleaning process to ensure that data collected is accurate and reliable.

FIG. 12 is a flow diagram illustrating an exemplary method in which data from a wearable device (i.e., raw accelerometer data, for example) is processed in accordance with the methods of the present invention to derive certain statistics regarding physical activity, such as steps and the specific level of user activity (sedentary activity, light-intensity activity, medium-to vigorous-activity, etc.).

FIGS. 13A, 13B, and 13C provide charts of ICD-10 (International Classification of Diseases) groups that may be used as a feature set.

FIGS. 14A, 14B, and 14C are tables showing cross-section data demographics.

FIG. 15 is a screenshot of an exemplary score (i.e., Aktivo Score®) that is tied to the digital biomarker and provided to a user via an interface of the digital health platform, wherein the score is generally indicative of the user's overall health, as determined by the systems and methods of the present disclosure.

FIG. 16 is a screenshot of an exemplary timeline of the user's score over a given period of time (i.e., days, weeks, months, etc.) that can be provided to the user.

FIG. 17 is a screenshot of an exemplary user interface associated with the digital health platform of the present disclosure, specifically showing a gamified format and features, such as scores and badges, that incentivize use by rewarding users for achieving personalized health milestones.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present invention is directed to a digital health platform for providing real-time personalized health risk assessments and recommended interventions for a user based on a unique analysis of data collected from a user's mobile and/or wearable devices.

The digital health platform is configured to collect, harmonize, and analyze lifestyle data (gathered in real-time from a user's mobile and/or wearable devices) to generate a digital biomarker that is indicative of the user's risk in developing a health condition and/or predictive of the user's morbidity and mortality risk of a health condition. The digital biomarker is generally a single metric, based on the combined impact of daily physical activities and sleep, that indicates a given user's health risk for developing chronic diseases. The platform essentially analyzes an individual's exercise, light physical activities, sedentary behavior, and sleep, into the single metric that correlates with their health status and longevity, further predicting morbidity and mortality risks based on lifestyle factors and utilizing the metric to forecast personalized health risks and outcomes.

The digital health platform utilizes data harmonization, which is an essential step in the data collection process, as it ensures concordance across different types of devices, enabling the generation of a truly representative digital biomarker for the user. The digital health platform gathers real-time data from smartphones and wearables, connecting seamlessly with popular brands like Apple Watch, Fitbit, Garmin, Oura Ring, and Whoop for automatic data transfer. The digital biomarker generated from this data can then be combined with sociodemographic population information, electronic health records, and other relevant data sources. This combined data is fed into a risk analytics engine, which uses advanced algorithms to predict personalized cross-sectional and longitudinal health risks and outcomes for non-communicable diseases with enhanced accuracy. More specifically, in an effort to continuously improve the accuracy of health risk predictions, machine learning (ML) tools are utilized to train the risk analytics engine, a model built on datasets from multiple data banks.

Accordingly, the digital health platform is able to effectively connect various metrics, such as physical activity with sleep quality and/or emotional well-being, which is essential for a comprehensive understanding of health and well-being. As a result, the digital health platform allows for more effective patient monitoring and may reduce the need in-person visits on an already overburdened healthcare system. Through data harmonization and the use of ML models, the personalized health risk and outcome predictions provided by the digital health platform are representative of every user, regardless of the data sources, making the model highly valuable for applications such as underwriting and population health studies. In turn, digital health platform is able to outperform other currently existing mHealth apps in predicting morbidities, co-morbidities, and mortality risk. It allows timely interventions for at-risk groups, improving individual overall health outcomes, and reducing long-term healthcare costs.

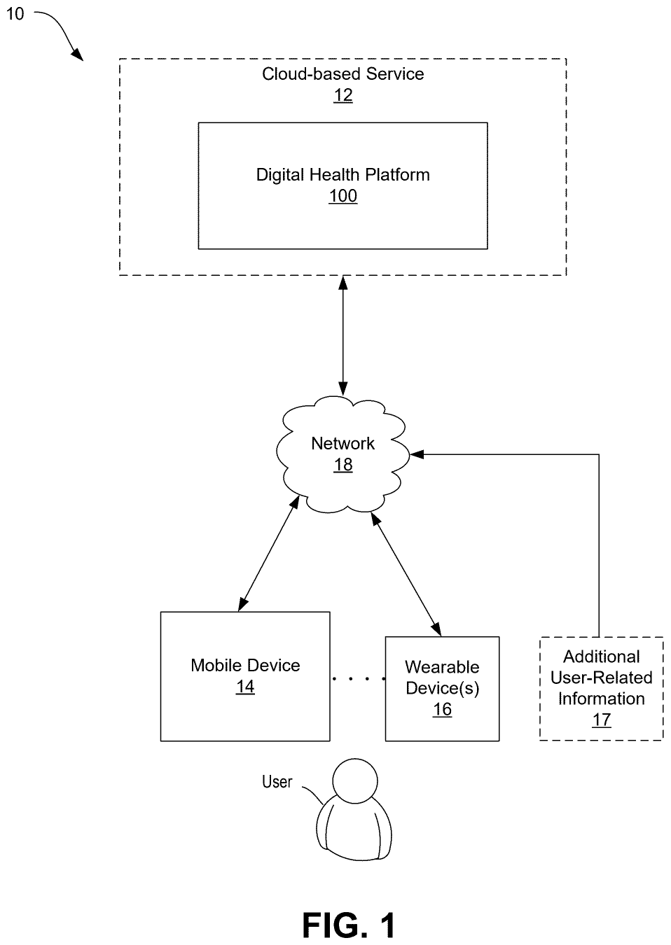

FIG. 1 is a block diagram illustrating one embodiment of an exemplary system 10 for providing real-time personalized health risk assessments and recommendations consistent with the present disclosure. As shown, the system 10 generally includes a digital health platform 100, which may be embodied on an internet-based computing system/service. For example, as shown, the platform 100 may be embodied on a cloud-based service 12, for example. The platform 100 is configured to communicate and share data, specifically health-related data, with one or more users via user mobile devices 14 over a network 18. The platform 100 is also configured to communicate and share data with wearable device(s) 16 that are generally associated with the user. The wearable devices 16 may include watches, wristbands, rings, and other forms wearable components that are configured to collect user-related data. The data collected from the mobile device 14 and wearable devices 16 is generally lifestyle-related data, including data associated with a user's sleep and physical activity. As such, the incoming data collected, in real-time, may include data associated with a user's movement and/or other user-related information, such as user sleep patterns, user vitals (heart rate, blood pressure, respiratory rate, temperature, etc.). Accordingly, such data can be passively collected from such devices.

It should further be noted that a user may also communicate with the platform 100 via a user-related computing device that may generally be embodied as a computer, a desktop computer, a personal computer (PC), a tablet computer, a laptop computer, a notebook computer, and the like. For example, as will be described in greater detail herein, a user may access an interface provided via the platform 100 either by way of their mobile device 14 or via a different user-related computing device (i.e., personal computer or the like). As such, in some instances, a user may provide additional user-related information 17 to the platform 100 (such as user name, date of birth, gender, height, weight, body mass index (BMI), user medical record data including medical history, comorbidity (ies), user self-assessment data associated with user sleep, user self-assessment data associated with user mental health, user diet data, etc.) via a computing device or their mobile device. In turn, the platform 100 is able to process/analyze such data to subsequently provide personalized health risk assessments and recommended interventions for a user.

The network 18 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the mobile device 14, the wearable devices 16, and the platform 100 may be, in whole or in part, a wired connection.

The network 18 may be any network that carries data. Non-limiting examples of suitable networks that may be used as network 18 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G), and future generations of cellular-based data communication technologies, Bluetooth radio, Near Field Communication (NFC), the most recently published versions of IEEE 802.11 transmission protocol standards, other networks capable of carrying data, and combinations thereof. In some embodiments, network 18 is chosen from the internet, at least one wireless network, at least one cellular telephone network, and combinations thereof. As such, the network 18 may include any number of additional devices, such as additional computers, routers, and switches, to facilitate communications. In some embodiments, the network 18 may be or include a single network, and in other embodiments the network 18 may be or include a collection of networks.

The platform 100 is configured to communicate and share data with a given user (i.e., a patient or individual interested in utilizing the health management services provided by the platform 100). Additionally, the platform 100 may be configured to communicate and share data with trusted individuals/entities that have permission/access rights to a given user's data, including a given user's personalized health risk assessments and recommended interventions. Such trusted individuals/entities may include a user's physician, clinician, medical facility, family members, etc.

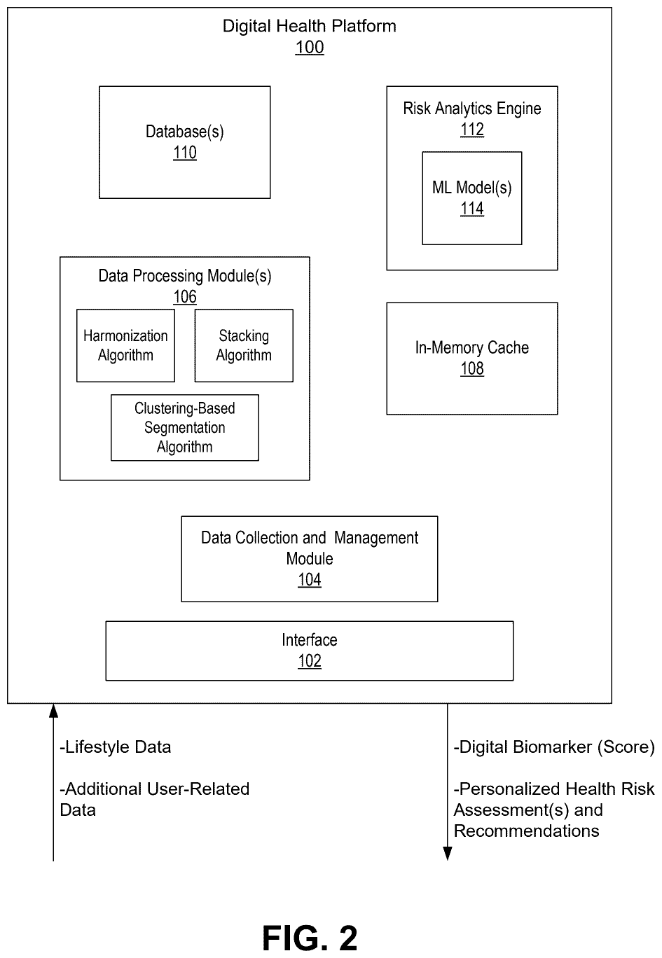

FIG. 2 is a block diagram illustrating the digital health platform 100 in greater detail. As shown, the platform 100 includes an interface 102, a data collection and management module 104, one or more data processing modules 106, in-memory cache 108, various databases 110 for storage of data, and a risk analytics engine 112. As will be described in greater detail herein, the various databases for storage of data may include a user database for storing profiles associated with each user, including, but not limited to, the specific lifestyle data collected from a given user's mobile device and/or wearable device, any other user-related information provided to, or collected by, the platform 100, any and all digital biomarker(s) calculated for the user at a given point in time based on the processing of such data, as well as any and all personalized health risk assessments and recommended interventions determined for a given user. The data collection and management module 104 may be configured to communicate and exchange data with each of the databases.

The interface 102 may generally allow a user (e.g., an authorized user) to access data on the digital health platform 100, via a mobile software application, for example, provided on a mobile device or via a web-based portal. For example, upon accessing a mobile software application, the interface 102 may be presented to the user via their device 14, in which the user may navigate a dashboard or standard platform interface so as to either initially register and create an account/profile so as to access the services provided by the platform 100 or to log in to access their account/profile. The interface 102 provides, among other things, personalized health assessments, daily lifestyle tracking, and engaging content. For example, the interface 102 may generally provide the digital biomarker in the form of a score, which simplifies a user's ability to evaluate their risk factors and overall health. The interface 102 may generally provide tools for self-monitoring and management, incorporating behavior change interventions, goal-setting, and gamification to enhance user engagement, which can further help motivate users and support better self-management of their chronic conditions.

The data collection and management module 104 is configured to receive session data (i.e., raw lifestyle data passively collected from a user's mobile device and/or wearable device, input from the user during a given session/interaction with the interface, including user selection data, etc.), at which point the data processing modules 106 are configured to process data (in conjunction with specific algorithms), wherein such processed data is to be analyzed, via the risk analytics engine 112, and a digital biomarker is calculated, wherein said digital biomarker is indicative of the user's risk in developing a health condition and/or predictive of the user's morbidity and mortality risk of a health condition. One or more data processing modules 106 may utilize the in-memory cache 108 and databases 112 during processing steps.

As shown, the platform 100 is configured to receive raw lifestyle data collected from different data sources associated with the user including at least a user's mobile device and/or one or more wearable devices associated with the user, the raw lifestyle data comprising data associated with sleep and physical activity of the user. The raw lifestyle data is then processed via one or more data processing modules 106. For example, the one or more data processing modules 106 are configured to process daily aggregates by running a harmonization algorithm to thereby standardize lifestyle data received from the different data sources and generate a structured timeline by merging the standardized lifestyle data from the different data sources. The one or more data processing modules 106 are further configured to process the structured timeline data by running a stacking algorithm to generate a structured, standardized set of daily aggregates comprising sleep data and physical activity time data associated with a specific level of user activity including: 1) sedentary-activity minutes; 2) light-intensity activity minutes; and 3) medium-to vigorous-activity minutes.

In turn, the risk analytics engine 112, running one or more machine learning (ML) models, is configured to analyze at least the structured, standardized set of daily aggregates of the user. The one or more ML models 114 have been trained using a plurality of training data sets, each training data set comprises reference sociodemographic data, reference lifestyle data, and reference health data associated with known health conditions. The platform 100 is configured to calculate, based on the analysis, a digital biomarker that is indicative of the user's risk in developing a health condition and/or predictive of the user's morbidity and mortality risk of a health condition. It should further be noted that platform 100 is configured to identify user behavior and match the user to closest peers by running a clustering-based segmentation algorithm. In turn, the platform 100 is configured to predict user behavior and determine recommended interventions identified as reducing risk in longitudinal timeframe.

In calculating the digital biomarker, and thus providing real-time personalized health risk assessments and recommended interventions, the platform utilizes artificial intelligence techniques (specifically utilizing the ML models 114, which may include, but are not limited to, a linear-based model, a tree-based model, and a neural network-based model.

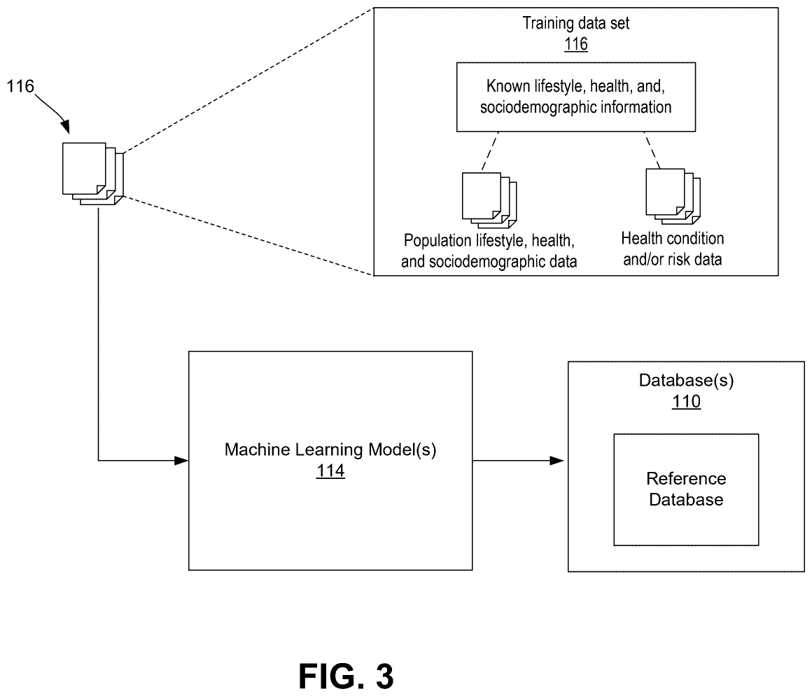

FIG. 3 is a block diagram illustrating inputting of reference data (i.e., training data sets) into one or more machine learning models consistent with the present disclosure. The machine learning techniques of the present invention, and the subsequent analysis and assessment of the incoming lifestyle data (having gone through a data harmonization process described herein), utilize reference data. The reference data may include a plurality of training data sets 116 inputted to a machine learning model 114 of the present invention. For example, each training data set includes reference population sociodemographic data, reference population lifestyle data, and reference population health data that has been associated with known health conditions and/or risk data.

The known health conditions may include but are not limited to: mental conditions or illnesses, such as depression; metabolic diseases such as diabetes; cardiovascular diseases such as hypertension, heart failure, transient ischemic attack (TIA) and coronary heath disease; stroke; and cerebrovascular diseases.

The reference data (i.e., population sociodemographic data, population lifestyle data, and population health data) and the associated known health conditions and/or risk data for each training data set may generally be obtained from one or more third-party sources, such as publicly available or subscription-based data sources. For example, reference data and the associated known health conditions and/or risk data may be obtained from a large-scale reputable biomedical database and research resource. In some embodiments, reference data and the associated known health conditions and/or risk data may be obtained from biobanks. In some embodiments, association of the reference data and the known health conditions and/or risk data is based, at least in part, on National Health and Nutrition Examination Survey (NHANES) guidance, for example.

The machine learning model 114 accesses reference data from the one or more training data sets 116 provided by any known source. The source may include, for example, a large-scale reputable biomedical database and research resource. Additionally, or alternatively, the source 300 may include publicly available registries and databases and/or subscription-based data sources. In preferred embodiments, the plurality of training data sets 116 feed into the machine learning model 114. The machine learning model 14 may include, but is not limited to, a linear-based model, a tree-based model, and a neural network-based model.

For example, in one embodiment, the machine learning model 114 may include a deep learning neural network that includes an input layer, a plurality of hidden layers, and an output layer. The autonomous machine learning model may represent the training data set using a plurality of features, wherein each feature comprises a feature vector. For example, the autonomous machine learning model may include a convolutional neural network (CNN). In other embodiments, the machine learning model 114 may include a recurrent neural network. The machine learning model 114 discovers associations in data from the training data sets. In particular, the machine learning model 114 processes and associates the reference population sociodemographic data, reference population lifestyle data, and reference population health data and health condition and/or risk data with one another, thereby establishing reference data in which known socioeconomic, lifestyle, and health data are associated with known health conditions and/or health risks. The reference data can be stored within a reference database, for example, in the databases 110, and available during subsequent processing of collecting data associated with a user undergoing analysis.

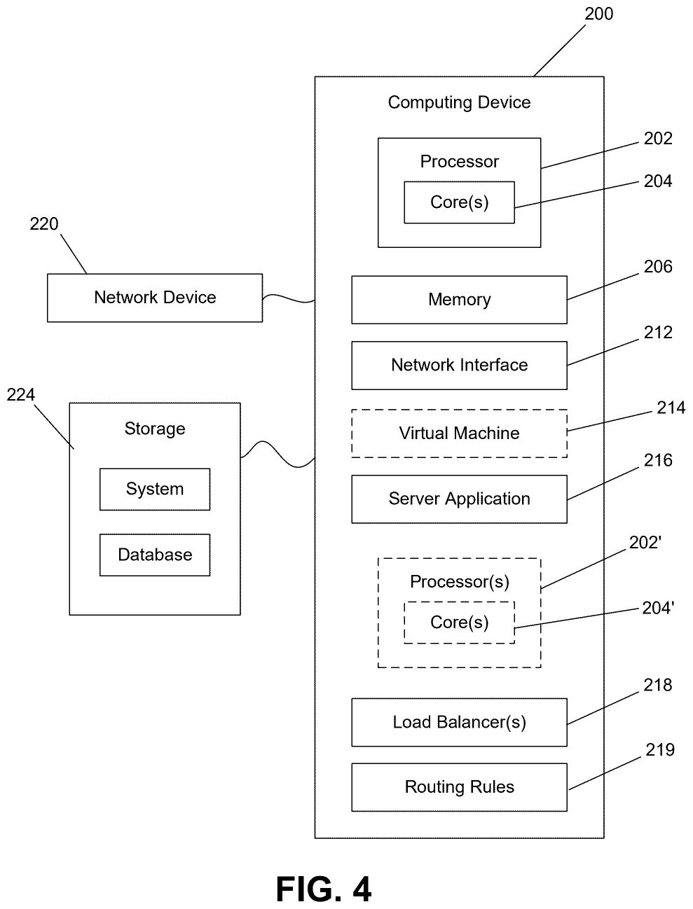

FIG. 4 is a block diagram illustrating an exemplary computing system 200 for implementing one or more servers for running the digital health platform 100 consistent with the present disclosure. In the present embodiment, the computing system 200 is configured as a server that is programmed and/or configured to execute one of more of the operations and/or functions for embodiments of the platform 100 described herein and to facilitate communication with the user devices via the user interface. The computing system 200 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more solid state drives), and the like. For example, memory 206 included in the computing system 200 can store computer-readable and computer-executable instructions or software for implementing exemplary embodiments of the components/modules of the platform 100 or portions thereof. The computing system 200 also includes configurable and/or programmable processor 202 and associated core 204, and optionally, one or more additional configurable and/or programmable processor(s) 202′ (e.g., central processing unit, graphical processing unit, etc.) and associated core(s) 204′ (for example, in the case of computer systems having multiple processors/cores), for executing computer-readable and computer-executable instructions or software stored in the memory 206 and other programs for controlling system hardware. Processor 202 and processor(s) 202′ may each be a single core processor or multiple core (204 and 204′) processor.

Virtualization may be employed in the computing system 200 so that infrastructure and resources in the computing device may be shared dynamically. One or more virtual machines 214 may be provided to handle a process running on multiple processors so that the process appears to be using only one computing resource rather than multiple computing resources. Multiple virtual machines may also be used with one processor.

Memory 206 may include a computer system memory or random access memory, such as DRAM, SRAM, EDO RAM, and the like. Memory 206 may include other types of memory as well, or combinations thereof.

The computing system 200 may include or be operatively coupled to one or more data storage devices 224, such as a hard-drive, CD-ROM, mass storage flash drive, or other computer readable media, for storing data and computer-readable instructions and/or software that can be executed by the processing device 202 to implement exemplary embodiments of the components/modules described herein with reference to the platform 100.

The computing system 200 can include a network interface 212 configured to interface via one or more network devices 220 with one or more networks, for example, a Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections (including via cellular base stations), controller area network (CAN), or some combination of any or all of the above. The network interface 212 may include a built-in network adapter, network interface card, PCMCIA network card, card bus network adapter, wireless network adapter, USB network adapter, modem or any other device suitable for interfacing the computing system 200 to any type of network capable of communication and performing the operations described herein. While the system device 200 depicted in block diagram is implemented as a server, exemplary embodiments of the computing system 200 can be any computer system, such as a workstation, desktop computer or other form of computing or telecommunications device that is capable of communication with other devices either by wireless communication or wired communication and that has sufficient processor power and memory capacity to perform the operations described herein.

The computing system 200 may run any server operating system or application 216, such as any of the versions of server applications including any Unix-based server applications, Linux-based server application, any proprietary server applications, or any other server applications capable of running on the computing system 200 and performing the operations described herein. An example of a server application that can run on the computing device includes the Apache server application.

The computing system 200 may further include one or more load balancers 218 for distributing network traffic across a group of resources to improve the efficiency of processing tasks. The one or more load balancers 218 may distribute network traffic in accordance with routing rules 219 so as to direct user traffic to the appropriate server or backend service to thereby improve efficiency and user experience.

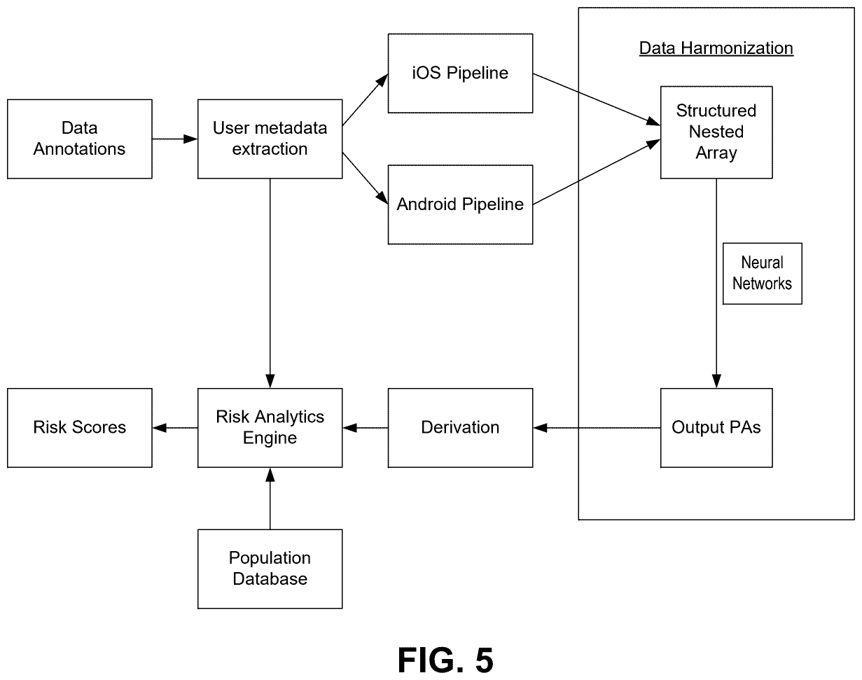

FIG. 5 is a flow diagram illustrating the overall process by which data is collected and analyzed to provide personalized health risks for a given user. As generally illustrated, the digital health platform is configured to collect, harmonize, and analyze lifestyle data (gathered in real-time from a user's mobile and/or wearable devices) to generate a digital biomarker that is indicative of the user's risk in developing a health condition and/or predictive of the user's morbidity and mortality risk of a health condition. The digital biomarker is generally a single metric, based on the combined impact of daily physical activities and sleep, that indicates a given user's health risk for developing chronic diseases. The platform essentially analyzes an individual's exercise, light physical activities, sedentary behavior, and sleep, into the single metric that correlates with their health status and longevity, further predicting morbidity and mortality risks based on lifestyle factors and utilizing the metric to forecast personalized health risks and outcomes.

The digital health platform utilizes data harmonization, which is an essential step in the data collection process, as it ensures concordance across different types of devices, enabling the generation of a truly representative digital biomarker for the user. The digital health platform gathers real-time data from smartphones (which can be running different operating systems, such as Android or iOS operating systems) and different wearables, connecting seamlessly with different types and brands of wearable devices, such as Apple Watch, Fitbit, Garmin, Oura Ring, and Whoop. The digital biomarker generated from this data can then be combined with sociodemographic population information, electronic health records, and other relevant data sources. This combined data is fed into a risk analytics engine, which uses advanced algorithms to predict personalized cross-sectional and longitudinal health risks and outcomes for non-communicable diseases with enhanced accuracy. More specifically, in an effort to continuously improve the accuracy of health risk predictions, machine learning (ML) tools are utilized to train the risk analytics engine, a model built on datasets from multiple data banks.

The digital health platform provides an interface with which a user can interact via their smartphone or personal computing device. The interface provides, among other things, personalized health assessments, daily lifestyle tracking, and engaging content to promote healthier habits, enabling individuals, corporations, and insurers to monitor health metrics effectively and make informed choices for long term well-being. For example, the interface may generally provide the digital biomarker in the form of a score, which simplifies a user's ability to evaluate their risk factors and overall health. The interface further supports self-reporting and empowers users to take preventive actions to manage their chronic disease risk. The platform interface may generally provide tools for self-monitoring and management, incorporating behavior change interventions, goal-setting, and gamification to enhance user engagement, which can further help motivate users and support better self-management of their chronic conditions. For example, the user interface may provide health scores, recommendations, and comparative analytics in a gamified format that incentivizes use by rewarding users for achieving personalized health milestones.

The present invention utilizes deep learning models to classify various physical activities (PAs) that users perform with their smartphones. The existing approach for PA recognition depends on predefined rules. In accordance with the present disclosure, the digital health platform makes use of deep neural networks to learn the patterns and features of PAs from the data to thereby achieve improved accuracy and robustness over typically used rule-based methods. Furthermore, the present invention is able to infer wearable data from smartphone data, thereby allowing the standardization of data across both devices. Generally, a wearable device tends to output greater values than a smartphone outputs for a given physical activity. For example, in the event a user has their phone in their pocket, and is also wearing a wearable device (such as a watch) while performing a given physical activity, the wearable device tends to output physical activity data that has a greater value than physical activity data outputted from the smartphone (i.e., the watch indicates that the user walked far greater steps than what the smartphone indicates, even though both were present for the same activity). As such, wearable users tend to have higher scores and metrics than phone users for the same activity.

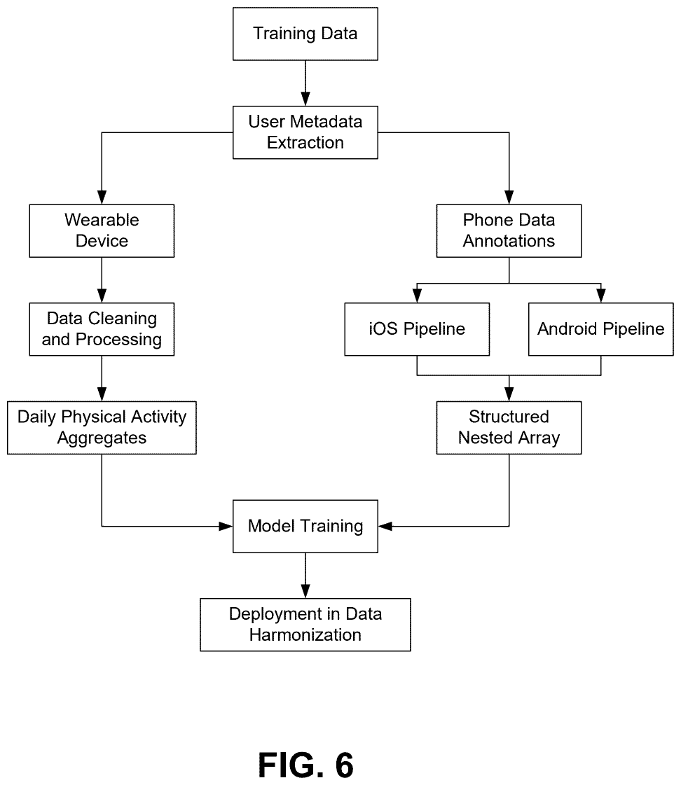

FIG. 6 is a flow diagram illustrating an exemplary process by which data is collected from mobile devices (i.e., smartphones) and wearable devices (i.e., watches, wristbands, rings, etc.) and subsequently processed for data harmonization purposes. The systems and methods of the present invention utilize deep learning models to accurately predict the wearable PAs from raw phone data, thereby resulting in harmonization of data, regardless of the source (smartphone or wearable). Raw data and PAs are passed to a data pipeline which transforms the data into structured data used in deep learning models. Sample transformed data is presented in Table 1, below. In the case of Android, hk_type and mm are nulls and in case of iOS, activity is null.

TABLE 1
Sample transformed data

| timestamp | steps | hk_type | activity | motionmanager |
|---|---|---|---|---|
| t | 2 | walking | NaN | walking |
| t + 1 | 10 | walking | NaN | Walking |
| t + 2 | 5 | walking | Nan | Walking |
| . . . | . . . | . . . | . . . | |
| . . . | . . . | . . . | . . . | |
| t + n | n | walking | NaN | walking |

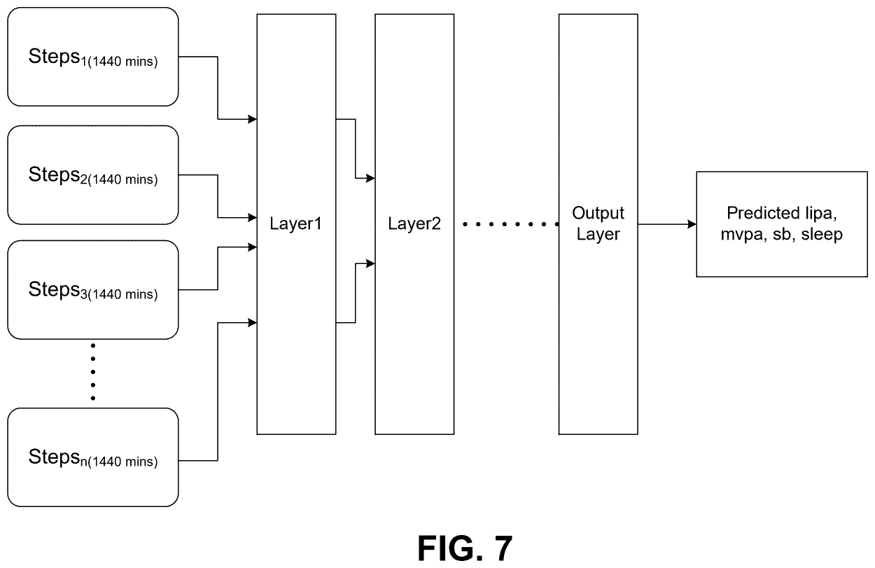

FIG. 7 shows an exemplary embodiment of a machine learning model used for predicting various physical activities that users perform (based on data harmonization techniques so as to standardize data across smartphones and wearable devices). Data is preprocessed to thereby remove noise and outliers, and then such data is fed to deep learning models. The models are trained on various features and parameters to optimize the accuracy and efficiency of the PA prediction. For example, the systems and methods of the present invention may utilize a Long Short Term Memory (LSTM) network, a type of recurrent neural network capable of learning order dependence in sequence prediction problems. In the present invention, LSTM is used to predict the PAs.

In an initial version of modelling, only step count per minute is used to predict the wearable PAs. Input feature to the model are number of steps per minute in a day and output is the respective PAs. In a second version of modelling, only steps data along with activities/hkworkouts recorded is used to predict the wearable PAs. Along with steps input additional feature, type of activity are added as feature. In a third version of modelling, only steps, activities/hkworkouts and motionmanager derived from iOS smartphones are used to predict the wearable PAs. Similar to activities motionmanager is added as an additional feature to model. In a fourth version of modelling, user metadata, such as age, gender, and BMI are used as input parameters, along with the features previously described herein.

Referring to FIG. 6, raw input data from smartphones is transformed into structured data that can be used for deep learning processes, including use for calculating a digital biomarker that is indicative of the user's risk in developing a health condition and/or predictive of the user's morbidity and mortality risk of a health condition.

The data pipeline transforms raw input data into structured data that can be used for deep learning purposes. Preprocessing of Android and iOS data points are different, as they have different features that need customized data preprocessing steps. This ensures that the deep learning models can handle both types of data. The data includes steps, activities, motion manager, etc. for each user. The aggregate data of each user for the same duration is collected and stored in blob. Then, the data is sorted into Android and iOS categories based on the parameters.

### Android Data Processing

Android data processing involves analyzing various types of information from users' smartphones, such as steps, activities, sleep, etc. However, only steps and activities are relevant for this task. The data is collected for a given date range and processed as follows:

Steps: This data shows the number of steps per minute that the user has taken. However, there may be duplicate records when the data spans over multiple days. Therefore, the data needs to be cleaned and filtered to remove any duplicates.
Activities: This data shows the type and duration of activities that the user has performed, such as walking, running, cycling, etc. However, the data may have different time intervals, such as 5 minutes or 10 minutes. Therefore, the data needs to be resampled to a uniform interval of 1 minute. Moreover, there may be duplicate records or missing values when the data is written over multiple days or when the device fails to capture the activities or the user is inactive. Therefore, the data needs to be cleaned and imputed to fill any gaps or remove any duplicates. Few instances where a single activity contains multiple sub activities in same duration of time.
Merge: After cleaning and resampling both steps and activities data, they need to be merged into a single dataset that contains both the number of steps and the type of activity for each minute for each user.

### iOS Data Processing

To analyze the data from IOS users, we need to consider three types of data: steps, hkworkouts and motionmanager. These data capture the number of steps per minute, the workouts and duration, and the movement detected by the device, respectively. Therefore, we need to perform the following steps:

### Steps

This data shows the number of steps per minute that the user has taken. However, there may be duplicate records when the data is written over multiple days. Therefore, the data needs to be cleaned and filtered to remove any duplicates.

### HKworkouts

This data shows the type and duration of activities that the user has performed, such as walking, running, cycling, etc. However, the data may have different time intervals, such as 5 minutes or 10 minutes. Therefore, the data needs to be resampled to a uniform interval of 1 minute. Moreover, there may be duplicate records or missing values when the data is written over multiple days or when the device fails to capture the activities or the user is inactive. Therefore, the data needs to be cleaned and imputed to fill any gaps or remove any duplicates. Few instances where a single activity contains multiple sub activities in same duration of time.

### Motionmanager:

Motionmanager collects data on the user's activity, such as whether they are unknown, stationary, walking, running, cycling or automative. The data may have duplicate or overlapping records if it written over multiple days, so it needs to be cleaned and filtered. The data also has variable time intervals, so it needs to be resampled to a consistent frequency of 1 minute. In few cases the activity is not detected and the values are marked as false.

### Merge:

After cleaning and resampling steps, hkworkouts and motionmanager data, they need to be merged into a single dataset that contains the number of steps, the type of activity for each minute and motion detected for each user.

The systems and methods of the present invention provide machine learning algorithms for forecasting wearable aggregates (PAs) utilizing raw smartphone data as an alternative to the current method.

Smartphone data, includes step count along with the type of activity, such as walking or running, recorded throughout the day. This data is typically logged in minute intervals (each day comprises of 1440 minutes). Approximately 90% of these data points are null. To address this, null values are substituted with zeros, under the assumption that during periods of inactivity or sleep, step counts would naturally be zero.

The respective wearable data consists of different activities (PAs) recorded in seconds. For example, in a single day, there might be 5000 seconds spent on Sleeping, 6000 seconds on Sedentary Behavior, 2000 seconds on low-intensity physical activity, and 1400 seconds on high-intensity physical activity.

Different machine learning algorithms have been used to model the data, including XGBoost, LightGBM, Decision Trees, Artificial Neural Networks, and Random Forests. The performance of these models is evaluated using the Root Mean Square Error (RMSE) metric. RMSE is used to measure how far off predictions are from the actual values.

The Random Forest model tends to outperform the other algorithms in the modeling, as indicated in Table 2 below:

TABLE 2
Performance of machine learning models versus existing methods

| | RMSE (Existing | RMSE (Model |
|---|---|---|
| Physical activity | method) | predictions) |
| SB (Sedentary behavior) | 2.55 | 2.11 |
| LIPA (Light intense activity) | 1.68 | 1.04 |
| MPA (High intense activity) | 1.03 | 0.86 |

The model achieved the lowest RMSE of 1.04 hours for LIPA, significantly surpassing the existing method's RMSE of 1.68 hours. Similarly, for MPA, the model's RMSE of 0.86 hours compares favorably to the existing method's 1.03 hours. Likewise, for SB, the model achieves an RMSE of 2.11, which is superior to the existing method's RMSE of 2.55.

Comparison of SB with Machine Learning Based Method Vs Existing Method

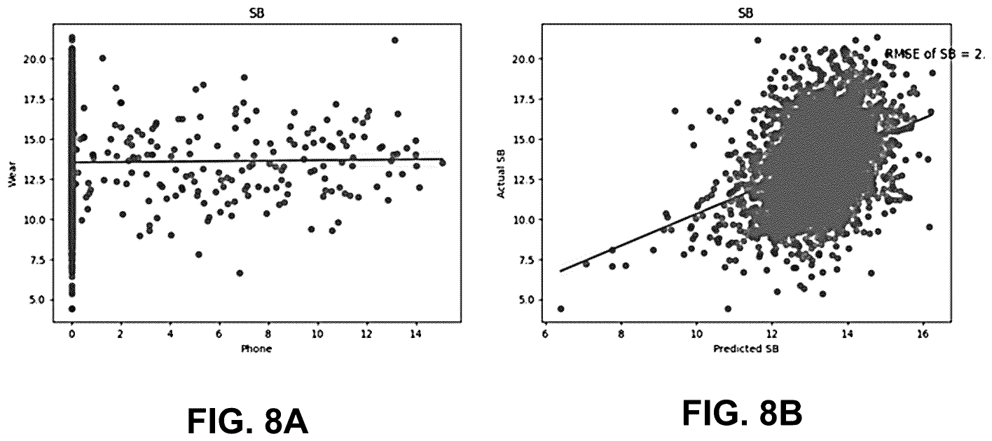

The phone and wear actual data demonstrates a high RMSE, showcase a larger predictive error, as shown in FIGS. 8A and 8B. The machine learning based model successfully captures the underlying trend, leading to a significant reduction in RMSE when compared to current method predictions.

Comparison of LIPA with Machine Learning Based Method Vs Existing Method

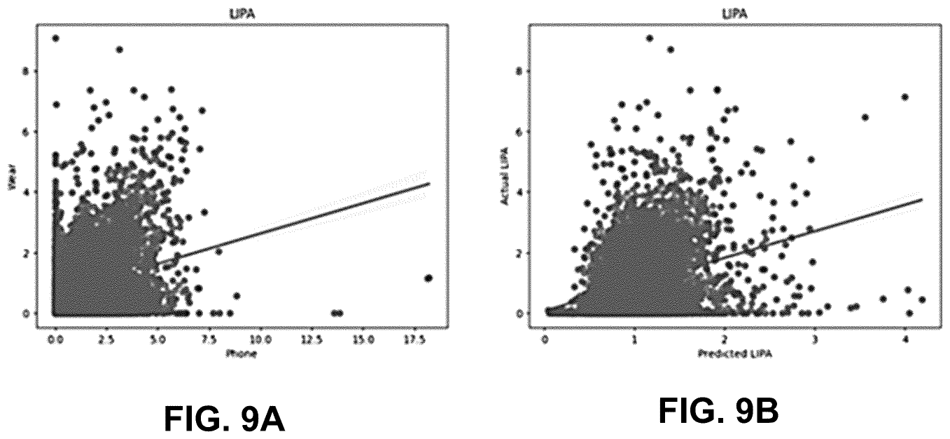

Referring to FIGS. 9A and 9B, the model effectively captured the overall trend. However, there are occasional instances where a horizontal line is formed parallel to the x-axis specifically at the actual 0 value level, which is probably due to a lack of variance in the dataset.

Comparison of MPA with Machine-Learning Based Method Vs Existing Method

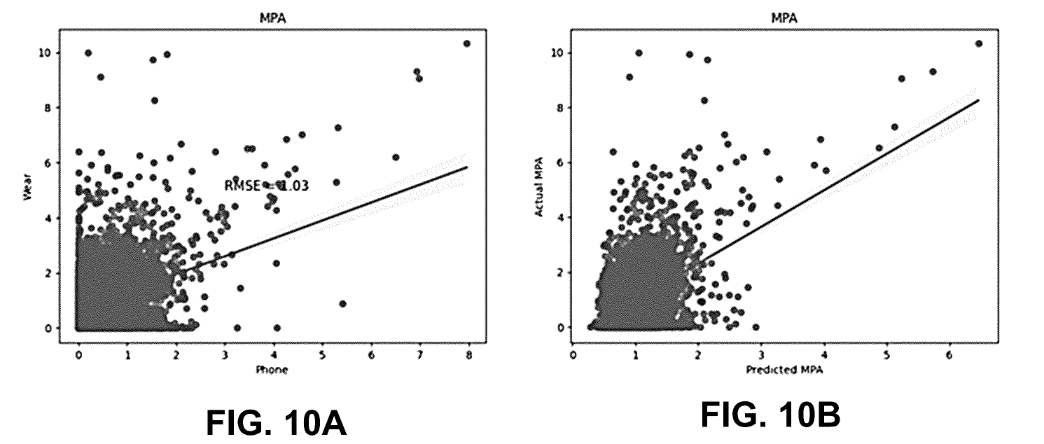

Referring to FIGS. 10A and 10B, the RMSE for the existing method is 1.03, while the model's RMSE stands at around 0.86, indicating an improved performance of the model in comparison to the current approach. The machine learning based model successfully captures the underlying trend and demonstrates better performance, leading to a significant reduction in RMSE when compared to the predictions made by the current method.

### Risk Analytics Engine

As previously described herein, the risk analytics engine is an ensemble of machine learning models that have been trained to predict mortality and various morbidity groups along various time frames, demographics, and geographical locations. Multiple models are created for each of the multiple feature sets for each target variable to build a robust and flexible engine. The initial version of the engine has been trained on publicly available datasets and subscribed biobank dataset which is a large-scale biomedical database with information of over half a million participants from UK. After data cleaning, there are approximately 92,000 participants data for which accelerometer data is present and their Aktivo Scores® can be derived. By including Aktivo Scores® in features, the models outperform state of the art methods in predicting morbidities, comorbidities and mortality risk. The risk analytics engine can be used to predict morbidity and mortality risk over real-time application users, client-side integrated users, as well as on any database of users with accelerometer data. The engine also contains longitudinal models that gives hazard ratios from cox models and accelerated failure time models.

The risk analytics engines support various risk categories, including, but not limited to, mortality risk, hospitalization risk, depression, diabetes type-2, heart failure, coronary heart disease, and transient ischemic attack. Along with risk, the engine can provide financial and propensity scores for the users as well, so as to drive hyper-personalization across product placement and user journey. The engine is built upon several data pipelines and architected with multiple models which enables it to run and learn from other datasets irrespective of the feature sets, geographical locations, and ethnicity. It also allows integration of other morbidity with relative ease.

### Data Cleaning

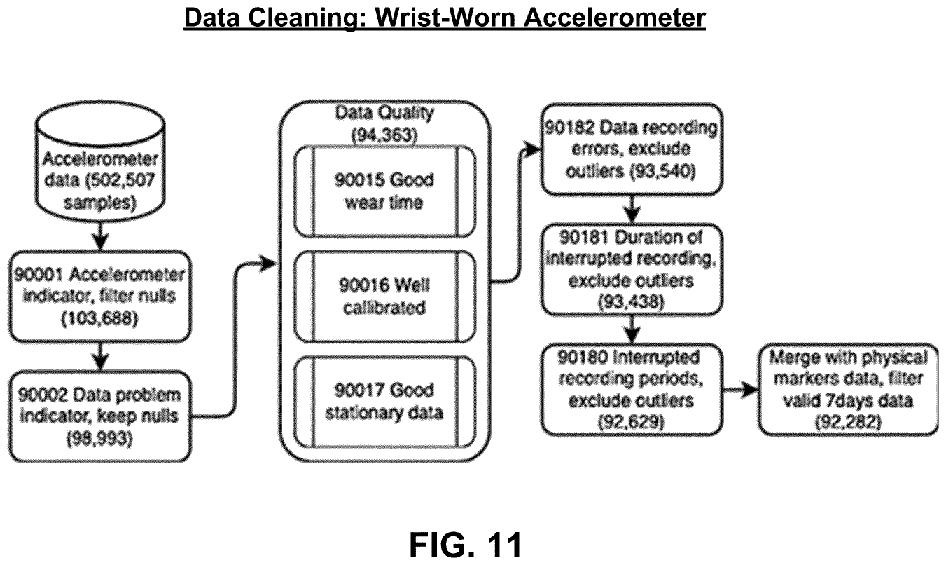

FIG. 11 is a flow diagram illustrating an exemplary data cleaning process to ensure that data collected is accurate and reliable. In some cases, the wrist-worn accelerometer did not report consistent or accurate recording or had large, interrupted periods of recordings. To make sure that the data used is accurate and reliable, the following steps were performed:
1. Total number of raw data points are 502,507.
2. Field 90001 is a Accelerometer indicator. Null value indicates there is no accelerometer data, if data is present value is recorded. After applying the filter, total number of samples are 103,688.
3. Field 90002 is a data problem indicator. Any value indicates the data recording is missing for that participant. After applying the filter, total number of samples are 98,993.
4. Fields 90015 (good wear time), 90016 (good calibration), 90017 (calibrated on own) indicate the data quality obtained from accelerometer. After applying the filter, total number of samples are 94,363.
5. Field 90182 indicates the data recording errors. To select only valid recordings we have discarded the outliers based on IQR (below 3quantile+1.5*IQR). After applying the filter, total number of samples are 93,540.
6. Field 90181 indicates the duration of interrupted recording. To select only valid recordings we have discarded the outliers based on IQR (below 3quantile+1.5*IQR). After applying the filter, total number of samples are 93,438.
7. Field 90180 indicates the Interrupted recording periods. To select only valid recordings we have discarded the outliers based on IQR (below 3quantile+1.5*IQR). After applying the filter, total number of samples are 92,629.
8. Combined with the physical markers such as sb, lipa etc. to keep only samples with a valid data for 7 days. After applying the filter, final number of samples are 92,282.

### Accelerometer Data Processing

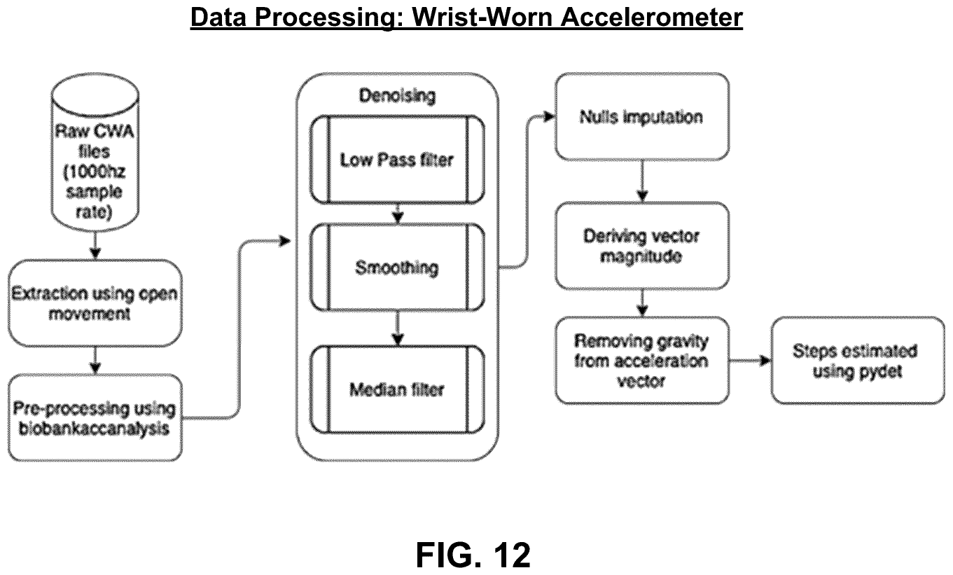

FIG. 12 is a flow diagram illustrating an exemplary method in which data from a wearable device (i.e., raw accelerometer data, for example) is processed in accordance with the methods of the present invention to derive certain statistics regarding physical activity, such as steps and the specific level of user activity (sedentary activity, light-intensity activity, medium-to vigorous-activity, etc.).

Participants wore a wrist-worn accelerometer for seven days that was posted to them. For deriving Aktivo Scores® of the participants, we processed raw accelerometer data as shown in FIG. 12 to derive stats such as steps, lipa, etc.
1. Raw “CWA” files containing triaxial accelerometer recordings from Axivity AX3 wrist worn accelerometers were acquired from the publicly available and subscribed biobank bulk datasets.
2. Axial acceleration information (1000 hz sample rate) was extracted from the raw CWA data using open movement's cwa-convert utility and parallelized using ray.
3. This was then pre-processed using the biobankaccanalysis package.
4. The data was down sampled to 100 hz using a 10-wide window mean.
5. Nulls were imputed using a forward fill and the vector magnitudes computed.
6. Denoising was then applied via a low pass filter (rate=100, freq=20 hz, order=4) followed by smoothing with a median filter (window_size=19) and the gravity removed from the acceleration vector.
7. Steps were then estimated from the normalized data via peak detection with the pydet package (threshold=0.21).

### Feature Sets

There are multiple feature sets that are designed for a particular model. It starts with the minimal number of features such as age, gender and Aktivo score® and moves on to include further features such as comorbidities, family histories, diet, mental health score, etc. The following table (Table 3) lists possible features:

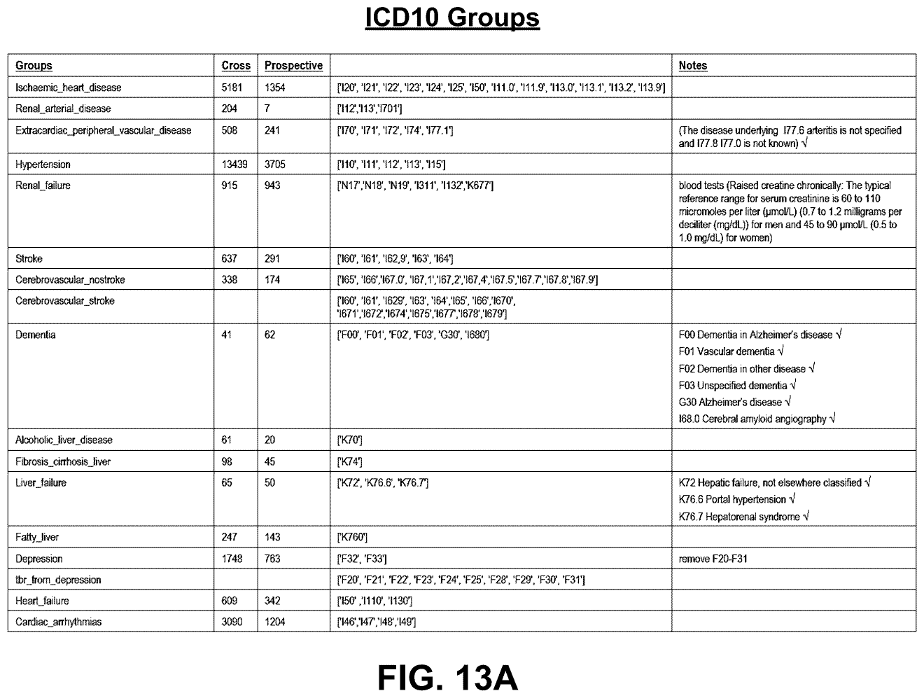

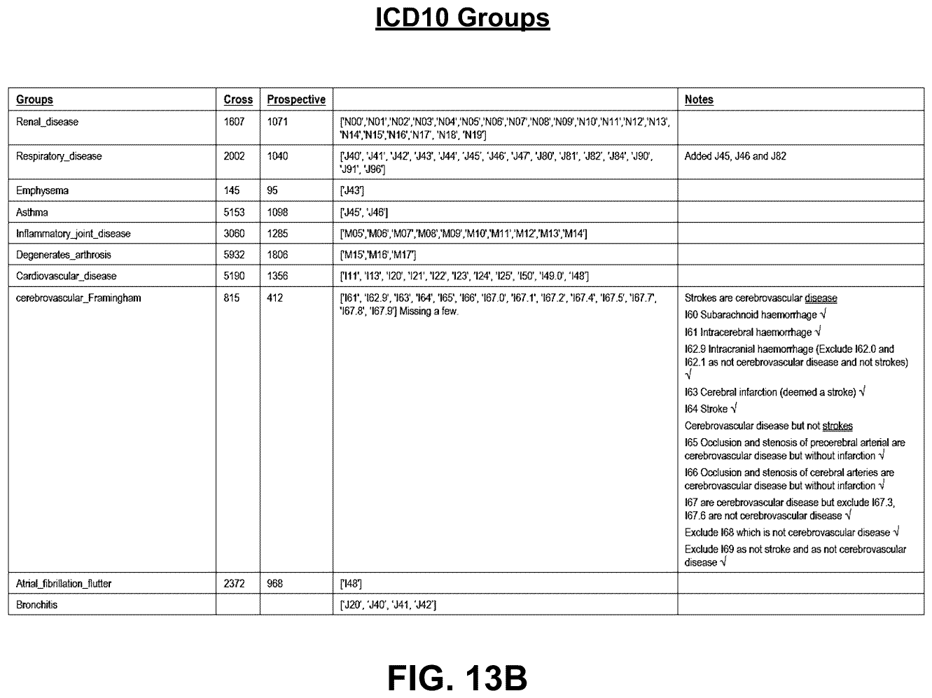

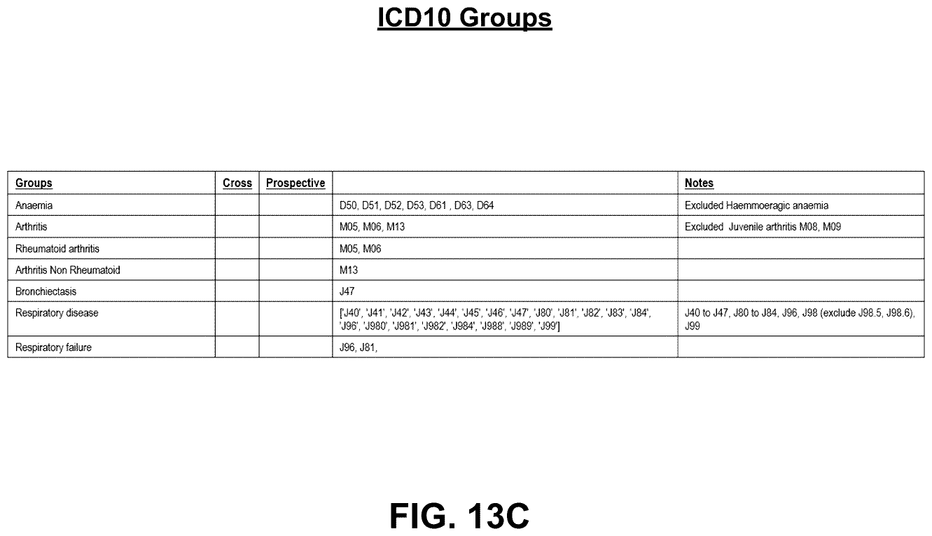

TABLE 3

| | | |
|---|---|---|
| Demographics | Age, Sex and bmi | Age is calculated from |
| | | birth to the date Aktivo |
| | | Score ® is recorded. |
| Physical | Aktivo Score ®, MVPA, | These stats are generated |
| Markers | reported sleep, LIPA, | through accelerometer |
| | Sedentary | data. Sleep is reported by |
| | | the participant in the |
| | | self-assessment |
| | | questionnaire |
| ICD10 Groups | See FIGS. 13A, 13B, and | The diagnosis are |
| | 13C. | captured from medical |
| | | records and classified into |
| | | the morbidity groups |
| Mental Health | Phq9 score, gad7 score, | Mental health scores are |
| | who5 score | calculated from self- |
| | | assessment of the |
| | | participants. |
| Diet | Ahei Scores: | We use 24 hour diet recall |
| | https://academic.oup.com/ | to calculate ahei score |
| | view-large/163466754 | which is a metric to rate |
| | | diet. |
| Medicines | These groups takes into | |
| | account any ongoing | |
| | medications for | |
| | hypertension, diabetes | |
| | and lipids. | |

Each features are checked for abnormal values and the nulls are replaced by mean in cases of linear models or kept as −1 for non-linear models.

### Machine Learning Models Used

### Logistic Regression

Logistic regression estimates the probability of an event occurring, such as voted or didn't vote, based on a given dataset of independent variables. Since the outcome is a probability, the dependent variable is bounded between 0 and 1. We train logistic regression after scaling and running Recursive Feature elimination to handle multicollinearity. The model is also regularized using Ridge regularization to prevent overfitting, i.e. learning of data instead of pattern by model.

### Random Forest

Random forest is an ensemble of large number of decision trees. Each individual tree in the random forest makes a class prediction and the class predicted by major trees becomes the whole models prediction. We train random forest with parameter tuning using Optuna.

### LightGBM

LightGBM is a gradient boosting framework that uses tree based learning algorithms. It is designed by Microsoft to be distributed and efficient with the following advantages: faster training speed and higher efficiency; lower memory usage; better accuracy; support of parallel, distributed, and GPU learning; and capable of handling large-scale data. Since lightgbm is prone to overfitting, multiple validation checks have been put to ensure that overfitting doesn't occur. Optuna is used for hyperparameter tuning for lightgbm.

### XGBoost

XGBoost is an optimized distributed gradient boosting library designed to be highly efficient, flexible and portable. It implements machine learning algorithms under the Gradient Boosting framework. XGBoost provides a parallel tree boosting (also known as GBDT, GBM) that solve many data science problems in a fast and accurate way.

Optuna is used for hyperparameter tuning.

### Model Types Based on Target

There are 4 scopes of models based on target variables:

### Cross Sectional Models

These are the models that are trained on risk profiles on the day of Aktivo Score® recorded. They predict risk of users on the time of Aktivo Score® being recorded. These models are trained on the correlation between the morbidity and the Aktivo Score®, depicting how similar a subjects movement is to diagnosed people with the same morbidity. In these models, if the subject has the specific morbidity on day of Aktivo Score® recorded, they are tagged as positive otherwise negative. Comorbidities, for it to be used as a feature, needs to happen at least 6 months before the target morbidity.

### 1-Year Models

These are the models that are trained on diagnosis within 1 year of Aktivo Score® recorded. They predict the risk of users for the next year after the Aktivo Score® is recorded. In these models, if the subject has the diagnosis record in the following 1 year of the Aktivo Score® recorded date, they are tagged as positive else negative. Users with diagnosis before Aktivo Score® date are removed from the dataset.

Comorbidities, for it to be used as a feature, needs to happen before the Aktivo Score® calculated date.

### 3-Year Models and 5-Year Models

These are same as 1 year models except the threshold is changed as per name.

### Hospitalization Models

These are the models that are trained on hospitalization of users excluding hospitalization due to accidents and infectious diseases. The models predict the risk of users to be hospitalized in the following 1, 3 and 5 years windows. These models can take in to account the current morbidities along with demographic feature of the users given the morbidities happen before the latest Aktivo Score is generated®. Hospitalization refers to inpatient records where the user spends at least 1 night admitted.

### Model Validation and Evaluation

All models, except when otherwise stated, were trained and evaluated using stratified k-fold cross validation with Performance measured on a hold out set. Stratification was performed using the target class to ensure consistent case-control proportions across all folds. This process has shown an optimistic result for imbalance classes. All models performance are captured by precision & recall for both classes. These would include specificity and sensitivity as recalls of positive and negative classes. F1 score are also captured.

However, the metric that is most significant is ROC-AUC score. The Receiver Operator Characteristic (ROC) curve is an evaluation metric for binary classification problems. It is a probability curve that plots the TPR against FPR at various threshold values and essentially separates the ‘signal’ from the ‘noise’. The Area Under the Curve (AUC) is the measure of the ability of a classifier to distinguish between classes and is used as a summary of the ROC curve.

The higher the AUC, the better the performance of the model at distinguishing between the positive and negative classes.

### Cross-Sectional Results

### Cross-Sectional Data Demographics

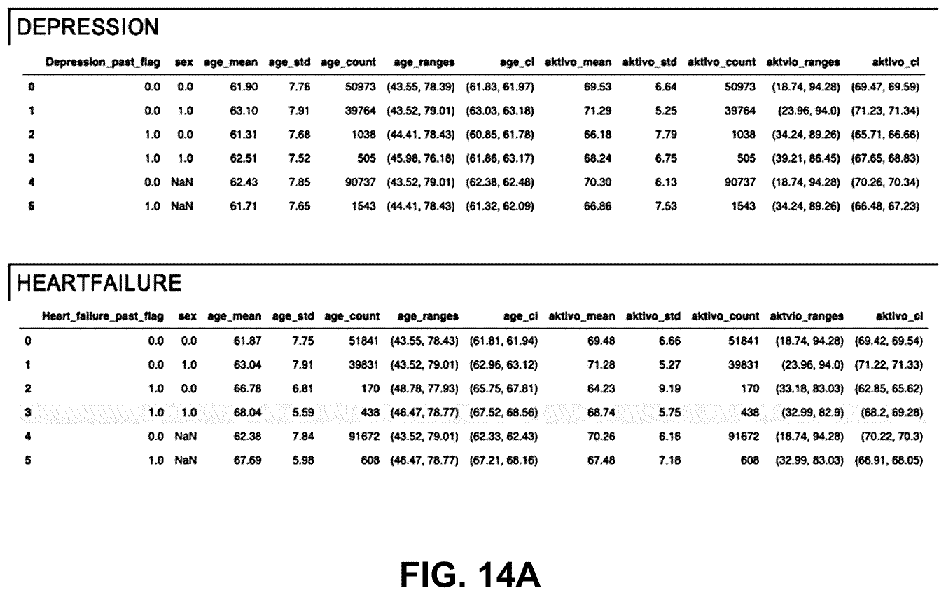

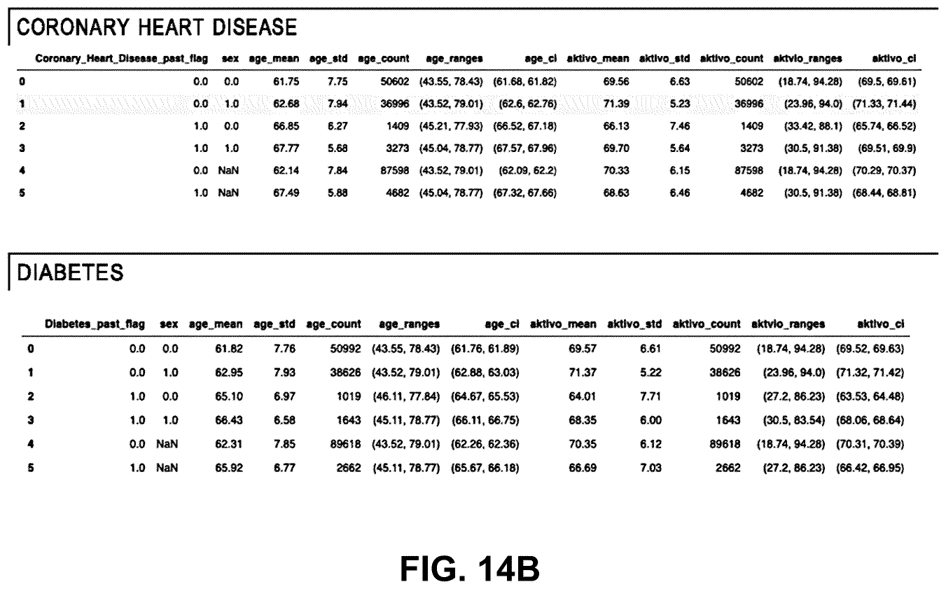

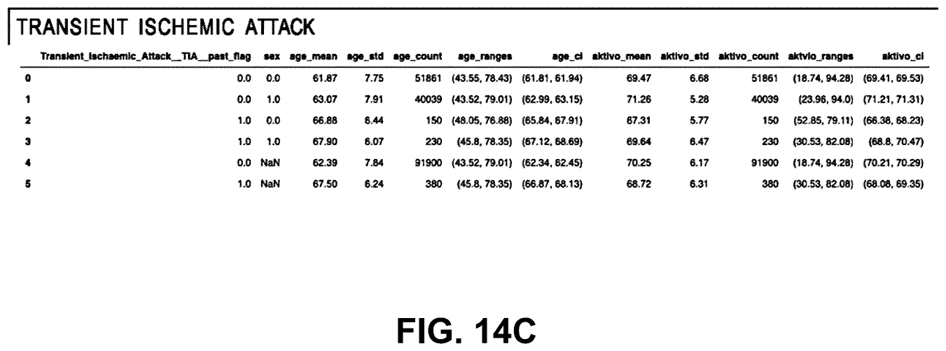

The tables shown in FIGS. 14A, 14B, and 14C show the distribution for the counts and ranges of Age and Aktivo Score® within morbidity groups and gender. Gender is denoted as: 1=male, 0=female.

### Model Results

The table below (Table 4) shows Model performance, captured in ROC-AUC for Base and Max feature sets. The base models consist of Aktivo Score®, Age, Sex and BMI as features. The Max models include all the features mentioned in Feature Sets, previously described herein.

TABLE 4
Model performance results

| Category | Base Model | Max Model |
|---|---|---|
| Depression | 0.690 | 0.850 |
| | (LogisticRegression) | (LogisticRegression) |
| Heart_failure | 0.754 (LightGBM) | 0.940 |
| | | (RandomForest) |
| Coronary_Heart_Disease | 0.784 | 0.890 (LightGBM) |
| | (LogisticRegression) | |
| Stroke | 0.650 (XGBoost) | 0.828 |
| | | (RandomForest) |
| Cerebrovascular_disease | 0.708 (LightGBM) | 0.909 |
| | | (LogisticRegerssion) |
| Diabetes | 0.808 (LightGBM) | 0.903 (LightGBM) |
| Transient_Ischaemic_Attack | 0.760 (XGBoost) | 0.830 (LightGBM) |
| Hypertension | 0.750 | 0.866 (LightGBM) |
| | (LogisticRegression) | |

Longitudinal Models

These are the time varying survival models developed over time to disease, i.e. the number of days to disease from the Aktivo Score® measurement. We use cox regression and AFT models for these and develop hazard ratio for the features. Since these models are very prone to non-convergence and can't handle multicollinearity, feature selection is done to ensure the most important features are included such as age, gender, Aktivo Score®, etc and other features are removed with p value greater than 0.05 to ensure only significant feature remains.

### Cox's Time Varying Proportional Hazard Model

Cox regression (or proportional hazards regression) is method for investigating the effect of several variables upon the time a specified event takes to happen. In the context of an outcome such as death this is known as Cox regression for survival analysis. Here apart from death we also use diagnosis of morbidities as the outcome with co-morbidities as variables.

Provided that the assumptions of Cox regression are met, this function will provide better estimates of survival probabilities and cumulative hazard than those provided by the Kaplan-Meier function.

The assumption we take for cox regression is that is that the hazards are proportional (PH), which means that the relative hazard remains constant over time with different predictor or covariate levels. However, we don't need to care about the PH assumptions due to large enough sample size. We can incorporate changes over time into our survival analysis by using a modification of the Cox model called Cox Time VaryingFitter.

### Accelerated Failure Time Model

AFT models are used in case PH assumption criteria is not satisfied. AFT is a parametric model which follows parametric continuous distributions like Weibull or Log-normal distribution. The model assumes that survival function that derived from two population are related by some acceleration factor which can be modelled as a function of covariates based on which the failure times can be accelerated or deaccelerated.

### User Interface

As previously described herein, the digital health platform provides an interface with which a user can interact via their smartphone or personal computing device. The interface provides, among other things, personalized health assessments, daily lifestyle tracking, and engaging content to promote healthier habits, enabling individuals, corporations, and insurers to monitor health metrics effectively and make informed choices for long term well-being.

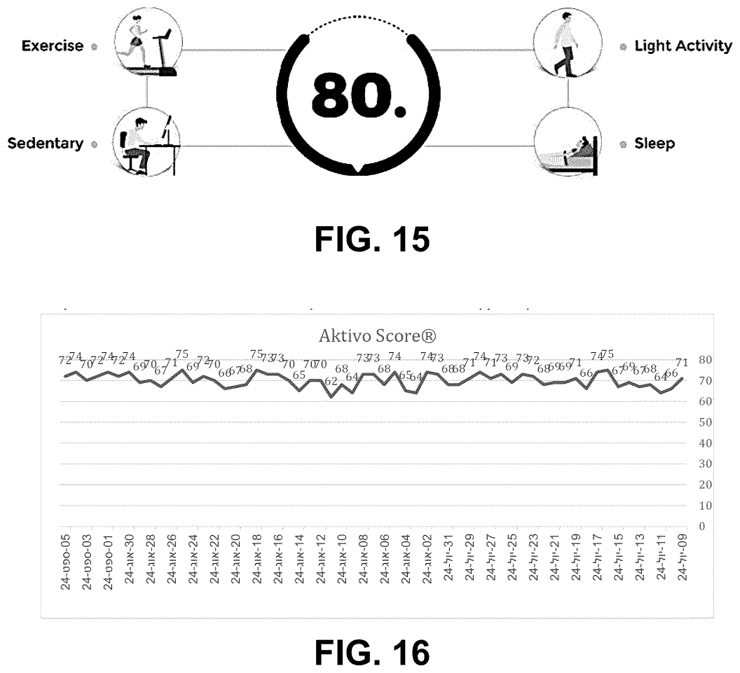

For example, the interface may generally provide the digital biomarker in the form of a score (i.e., an Aktivo Score®), which simplifies a user's ability to evaluate their risk factors and overall health. FIG. 15 is a screenshot of an exemplary score (i.e., Aktivo Score®) that is tied to the digital biomarker and provided to a user via an interface of the digital health platform, wherein the score is generally indicative of the user's overall health, as determined by the systems and methods of the present disclosure. The score can generally have a numerical value ranging from 1 to 99. When the value is below a predefined threshold, the user's risk in developing a health condition increases and when the value is above a predefined threshold, the user's risk in developing a health condition decreases. Accordingly, in the event that the value is below the predefined threshold, the platform is configured to generate and provide scientific feedback to motivate the user to increase the value of their score. The scientific feedback may include recommendations for increasing sleep and/or physical activity of the user. The platform is configured to provide real-time health recommendations and feedback to the user based, at least in part, on ongoing data collection and analysis. As such, a user can receive a score daily, and the interface provides tools allowing for the user to track their score over time and see trends. For example, FIG. 16 is a screenshot of an exemplary timeline of the user's score over a given period of time (i.e., days, weeks, months, etc.) that can be provided to the user.

The interface further supports self-reporting and empowers users to take preventive actions to manage their chronic disease risk. The platform interface may generally provide tools for self-monitoring and management, incorporating behavior change interventions, goal-setting, and gamification to enhance user engagement, which can further help motivate users and support better self-management of their chronic conditions. For example, the user interface may provide health scores, recommendations, and comparative analytics in a gamified format that incentivizes use by rewarding users for achieving personalized health milestones. The use of gamified features, such as scores and badges, may help users better interpret health data and manage their conditions, thereby improving their understanding of health signals and enhances adherence to healthy lifestyle and treatment plans.

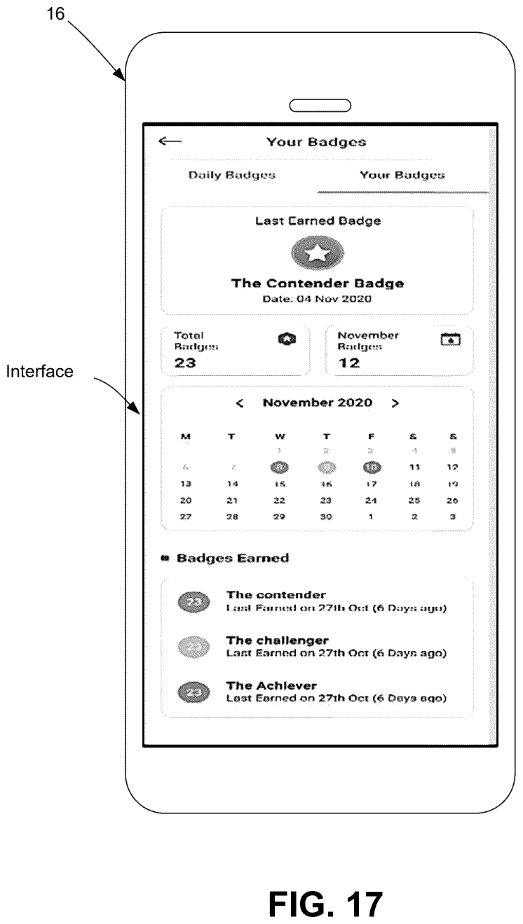

FIG. 17 is a screenshot of an exemplary user interface associated with the digital health platform of the present disclosure, specifically showing a gamified format and features, such as scores and badges, that incentivize use by rewarding users for achieving personalized health milestones.

Accordingly, the digital health platform is able to effectively connect various metrics, such as physical activity with sleep quality and/or emotional well-being, which is essential for a comprehensive understanding of health and well-being. As a result, the digital health platform allows for more effective patient monitoring and may reduce the need in-person visits on an already overburdened healthcare system. Through data harmonization and the use of ML models, the personalized health risk and outcome predictions provided by the digital health platform are representative of every user, regardless of the data sources, making the model highly valuable for applications such as underwriting and population health studies In turn, digital health platform is able to outperform other currently existing mHealth apps in predicting morbidities, co-morbidities, and mortality risk. It allows timely interventions for at-risk groups, improving individual overall health outcomes, and reducing long-term healthcare costs.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing real-time personalized health risk assessments, the system comprising:
a cloud-based digital health platform configured to communicate with one or more user-associated computing devices or software-agent-associated computing devices over a network, the digital health platform comprising a hardware processor coupled to non-transitory, computer-readable memory containing instructions executable by the processor to cause the platform to:
receive raw lifestyle data collected from different data sources associated with the user including at least a user's mobile device and/or one or more wearable devices associated with the user, the raw lifestyle data comprising data associated with sleep and physical activity of the user;
process the lifestyle data, including processing daily aggregates by running a harmonization algorithm to thereby standardize lifestyle data received from the different data sources and generate a structured timeline by merging the standardized lifestyle data from the different data sources;
process the structured timeline data by running a stacking algorithm to generate a structured, standardized set of daily aggregates comprising sleep data and physical activity time data associated with a specific level of user activity including: 1) sedentary-activity minutes; 2) light-intensity activity minutes; and 3) medium-to vigorous-activity minutes;
analyze, via a risk analytics engine running one or more machine learning (ML) models, at least the structured, standardized set of daily aggregates of the user, wherein the one or more ML models have been trained using a plurality of training data sets, each training data set comprises reference sociodemographic data, reference lifestyle data, and reference health data associated with known health conditions; and
calculate, based on the analysis, a digital biomarker that is indicative of the user's risk in developing a health condition and/or predictive of the user's morbidity and mortality risk of a health condition.

2. The system of claim 1, wherein the platform is further configured to identify user behavior and match the user to closest peers by running a clustering-based segmentation algorithm.

3. The system of claim 2, wherein the platform is further configured to predict user behavior and determine recommended interventions identified as reducing risk in longitudinal timeframe.

4. The system of claim 1, wherein the known health conditions comprise mental and physical health conditions.

5. The system of claim 4, wherein the known health conditions comprise non-communicable diseases.

6. The system of claim 4, wherein the known health conditions are selected from the group consisting of: mental illnesses such as depression; metabolic diseases such as diabetes; cardiovascular diseases such as hypertension, heart failure, transient ischemic attack (TIA) and coronary heath disease; stroke; and cerebrovascular diseases.

7. The system of claim 1, wherein the platform is configured to transmit, to a user-associated computing device, the digital biomarker to be presented to the user via a display on the user-associated computing device.

8. The system of claim 7, wherein the digital biomarker is a numerical value ranging from 1 to 99.

9. The system of claim 8, wherein:
when the value of the digital biomarker is below a predefined threshold, the user's risk in developing a health condition increases; and
when the value of the digital biomarker is above a predefined threshold, the user's risk in developing a health condition decreases.

10. The system of claim 9, wherein, when the value of the digital biomarker is below the predefined threshold, the platform is configured to generate and provide scientific feedback to motivate the user to increase the value of the digital biomarker.

11. The system of claim 10, wherein said scientific feedback comprises recommendations for increasing sleep and/or physical activity of the user.

12. The system of claim 1, wherein the mobile device comprises a smartphone and the one or more wearable devices comprise a watch or fitness tracker device, and wherein the raw lifestyle data comprises physical activity associated with at least one of user movement.

13. The system of claim 12, wherein the user activity is captured by a motion sensor, wherein the motion sensor comprises at least one of an accelerometer, one or more gyroscopes, and a magnetometer for capturing motion of the smartphone and/or the one or more wearable devices to thereby provide corresponding cadence of the user.

14. The system of claim 12, wherein the raw lifestyle data comprises one or more sets of step counts logged in minute intervals and a corresponding physical activity for each of the one or more step counts, the physical actively being selected from the group consisting of sleep, sedentary activity, low-intensity activity, and high-intensity activity.

15. The system of claim 14, wherein running a harmonization algorithm comprises running a neural network, wherein the neural network has been trained using a plurality of training data sets, each training data set comprises reference step count data associated with known physical activity from corresponding smartphones and wearable devices running different operating systems and/or platforms.

16. The system of claim 15, wherein the neural network comprises a deep learning neural network that includes an input layer, a plurality of hidden layers, and an output layer.

17. The system of claim 16, wherein each training data set is represented using a plurality of features, wherein each feature comprises a feature vector.

18. The system of claim 17, wherein the neural network comprises a recurrent neural network (RNN) with a combination of aggregating model using model stacking.

19. The system of claim 15, wherein processing the lifestyle data by running a harmonization algorithm and a stacking algorithm to generate a structured, standardized set of daily aggregates ensures concordance across different types of devices.

20. The system of claim 1, wherein the one or more ML models is selected from the group consisting of a linear-based model, a tree-based model, and a neural network-based model.

21. The system of claim 1, wherein the risk analytics engine is configured to analyze the structured, standardized set of daily aggregates of the user and other user-related information.

22. The system of claim 21, wherein said other user-related information comprises at least one of user gender, user age, user body mass index (BMI), user medical record data, user self-assessment data associated with user sleep, user self-assessment data associated with user mental health, and user diet data.

23. The system of claim 1, wherein at least some of the reference lifestyle data and the associated known health conditions for each training data set, of the plurality of training sets of the one or more ML models, are obtained from one or more third-party sources.

24. The system of claim 23, wherein the one or more third-party sources comprise publicly available or subscription-based data sources.

25. The system of claim 23, wherein at least some of the reference lifestyle data and the associated known health conditions are obtained from a large-scale reputable biomedical database and research resource.

26. The system of claim 25, wherein the at least some of the reference lifestyle data and the associated known health conditions are obtained from biobanks.

27. The system of claim 23, wherein association of the reference lifestyle data and the known health conditions are based, at least in part, on National Health and Nutrition Examination Survey (NHANES) guidance.

28. The system of claim 1, wherein the platform is configured to provide real-time health recommendations and feedback to the user based, at least in part, on ongoing data collection and analysis.

29. The system of claim 1, wherein the platform is configured to maintain end-to-end encryption of all user-related data collected and stored in compliance with data privacy regulations such as GDPR, HIPAA, or equivalent.

30. The system of claim 1, wherein the system is configured to integrate and/or communicate with a plurality of third-party health and fitness applications to import additional user-related data such as dietary habits and blood pressure.

31. The system of claim 1, further comprising a user interface configured to provide health scores, recommendations, and comparative analytics in a gamified format that incentivizes use by rewarding users for achieving personalized health milestones.

32. The system of claim 31, wherein the gamified format comprises a reward-based format including challenges, in which users are rewarded for achieving consistent improved scores.

33. The system of claim 1, wherein the platform provides multilingual support to ensure accessibility for users across different regions and languages.
